(19) United States
(12) Patent Application Publication
(10) Pub. No.: US 2019/0115099 A1
(43) Pub. Date: Apr. 18, 2019

(54) SYSTEMS AND METHODS FOR PROVIDING RESOURCE MANAGEMENT ACROSS MULTIPLE FACILITIES

(71) Applicant: Timothy Gayle Goux, Mandeville, LA (US)

(72) Inventor: Timothy Gayle Goux, Mandeville, LA (US)

(21) Appl. No.: 16/152,409

(22) Filed: Oct. 4, 2018

Related U.S. Application Data
(60) Provisional application No. 62/568,113, filed on Oct. 4, 2017.

(51) Int. Cl. G16H 40/20; G16H 10/60; G06Q 10/06; H04L 9/06

(52) U.S. Cl. CPC G16H 40/20; G16H 10/60; H04L 2209/38; H04L 9/0637; G06Q 10/0631

(57) ABSTRACT

A system including: at least one processor; and at least one memory having stored thereon computer program code that, when executed by the at least one processor, controls the at least one processor to: provide, to a resource acquisition system, information corresponding to at least one available resource; receive an indication of a selection of a resource from among the at least one available resource; and in response to receiving the selection, reserve the selected resource with a facility resource system and automatically schedule one or more auxiliary resources for the selected resource

### CROSS-REFERENCES TO RELATED APPLICATIONS

This application claims priority from U.S. Provisional Application No. 62/568,113, filed Oct. 4, 2017, the subject matter of which is incorporated herein by reference in its entirety.

### TECHNICAL FIELD

Aspects of the present disclosure relate to systems and methods for providing resource management, and, more particularly, to providing resource management across multiple facilities.

### BACKGROUND

Resource management is a consistent problem. For example, with today's ever-changing regulations and demands on healthcare providers, hospitals must discharge patients as soon as reasonable to the most ideal post-acute care setting to continue to receive the care that one may require. However, related art approaches are inefficient, causing resources to be unused and requiring multiple manual rounds of communication. Moreover, the varying rules for patients, insurance, and facilities creates many opportunities for failures in the related art. This places significant pressure on the hospital's discharge planning departments and staff.

In the related art, the discharge planning departments at hospitals spend hours calling all potential post-acute care providers to acquire information on availability and determine feasibility. Once determined, there routinely are multiple follow-up calls, email-fax communications that are centered around each discharge. Such communications are cumbersome and fail to provide sufficient security.

Accordingly, there is a need for improved systems and methods for providing resource management across multiple facilities. Embodiments of the present disclosure are directed to this and other considerations.

### SUMMARY

According to some embodiments, there is provided a system including: at least one processor; and at least one memory having stored thereon computer program code that, when executed by the at least one processor, controls the at least one processor to: provide, to a resource acquisition system, information corresponding to at least one available resource; receive an indication of a selection of a resource from among the at least one available resource; and in response to receiving the selection, reserve the selected resource with a facility resource system and automatically schedule one or more auxiliary resources for the selected resource.

The computer program code, when executed by the at least one processor, may further control the at least one processor to: receive, from the resource acquisition system, one or more resource requirements; and generate a facility listing of one or more facilities having an available resource matching the one or more resource requirements.

The one or more resource requirements may include at least one from among a resource type, facility location, facility type, rating, and transport services.

The one or more resource requirements may include a needed service, and the auxiliary resource may include at least one of a specialized device and a specialized staff.

The needed service may include dialysis, and the auxiliary resource includes a dialysis technician and a dialysis machine.

The computer program code, when executed by the at least one processor, may further control the at least one processor to receive, from the resource acquisition system, a required service. Automatically scheduling one or more auxiliary resources may include: identifying one or more specialized staff necessary for the required service; accessing an employee scheduling platform of the facility resource system; and scheduling the identified specialized staff to coincide with the selected resource reservation.

The computer program code, when executed by the at least one processor, may further control the at least one processor to: provide the information corresponding to the at least one available resource to a user device associated with a user requiring the resource; and receive the indication of the selection of the resource from the user device.

The computer program code, when executed by the at least one processor, may further control the at least one processor to modify, within the facility resource system, an availability indicator of the scheduled resource and auxiliary resources at the facility resource system.

According to some embodiments, there is provided a system including: at least one processor; and at least one memory having stored thereon computer program code that, when executed by the at least one processor, controls the at least one processor to: maintain a blockchain including facility data of a plurality of facilities; receive, from a resource acquisition system, a request for a healthcare resource; access the facility data to identify one or more facilities from among the plurality of facilities capable of providing the requested healthcare resource; receive an indication of a request to reserve the healthcare resource at a first facility among the one or more facilities; and update the facility data within the blockchain to identify the healthcare resource at the first facility as unavailable.

The blockchain may further store reservation information and the request to reserve the healthcare resource may include an indication of a patient. The computer program code, when executed by the at least one processor, may further control the at least one processor to update the reservation information within blockchain to indicate that the patient has reserved the healthcare resource at the first facility.

The reservation information may include a plurality of identifiers indicative of the patient, the first facility, and the reserved healthcare resource.

The blockchain may store patient health information of a patient a healthcare facility associated with the resource acquisition system. The computer program code, when executed by the at least one processor, may further control the at least one processor to: receive, from the healthcare facility, a discharge order for the patient; and automatically identify the one or more facilities from among the plurality of facilities capable of providing the requested healthcare resource based on the discharge order.

The computer program code, when executed by the at least one processor, may further control the at least one processor to update the blockchain to automatically identify the one or more facilities based on the discharge order by identifying, within the discharge order, a plurality of criteria.

The plurality of criteria may include at least one from among a geographic region, an accepted insurance, a needed service, and needed dates.

According to some embodiments, there is provided a system including: at least one processor; and at least one memory having stored thereon computer program code that, when executed by the at least one processor, controls the at least one processor to: identify, at a first healthcare facility, patient data corresponding to a first admitted patient; analyze the patient data to determine that the patient is to be discharged from the first healthcare facility and to identify a healthcare resource for the patient post-discharge; determine a second healthcare facility capable of providing the identified healthcare resource and having capacity for the patient; and schedule transfer of the patient from the first healthcare facility the second healthcare facility.

The computer program code, when executed by the at least one processor, may further control the at least one processor to identify a discharge order for the patient from within the patient data, and process the discharge order to determine the healthcare resource.

The computer program code, when executed by the at least one processor, may further control the at least one processor to process the patient data to determine a patient recovery trajectory and determine the healthcare resource prior to receiving a discharge order.

Scheduling the transfer of the patient may include reserving the identified healthcare resource at the second healthcare facility for the patient. Scheduling the transfer of the patient may include accessing a staff scheduling platform hosted by the second healthcare facility, and scheduling one or more staff members at the second healthcare facility to during the transfer. Scheduling the transfer of the patient may include accessing a staff scheduling platform hosted by the second healthcare facility, and assigning one or more healthcare machines required healthcare resource to the patient during the transfer.

These and further objects and advantages will be apparent to those skilled in the art in view of the detailed description set forth below.

### BRIEF DESCRIPTION OF THE FIGURES

Reference will now be made to the accompanying figures and flow diagrams, which are not necessarily drawn to scale, and wherein:

FIG. 1 illustrates an example system environment for implementing one or more aspects of the present disclosure.

FIG. 2 is a block diagram of an example computer system capable of implementing certain aspects of the present disclosure.

FIGS. 3-6 illustrate example interfaces of a software solution according to an example embodiment.

FIGS. 7-9 are flowcharts of example methods according to the present disclosure.

### DETAILED DESCRIPTION

In certain implementations, the disclosed system, which may be referred to as “CR” or “CR solution”, may provide a web based software solution that improves efficiency in the acute and post-acute healthcare space (e.g., hospitals, skilled nursing and rehab facilities, home health agencies, assisted and independent living facilities, retirement communities, hospice agencies) and for consumers.

The CR web-enabled software solution may allow healthcare providers to display their real-time availability to any and all referral sources (eliminating the need for multiple calls, etc.)—referral sources being hospitals, other healthcare providers, and consumers. The CR web-enable software solution may enable the display of accreditation scores and other ratings from both public and private sources. For example, and CR solution may obtain publicly available accreditation scores and ratings and score them in a database. The system may associate the scores and ratings stored in the database with particular healthcare providers and the system may display one or more scores or ratings on a webpage associated with the healthcare provider. The CR solution may allow for each healthcare provider to customize any of the information about their facility, including information such as bed types, care services offered, photographic images, the ability to reserve a bed/unit directly from the software application, convey estimated time of arrival, convey potential payor types, submit and upload pertinent medical record information through CR's encrypted technology, and display any rating the facility may want to present, such as ratings from regulatory or private agencies.

The CR solution may be made available to referral sources by the CR facility through secure access. Consumers may have access to the CR solution if the CR client selects that option.

For example, once a CR reservation is made by a hospital, all details and characteristics of that reservation may be transmitted to the CR client healthcare provider, which may include characteristics such as: bed type desired, specific care services needed, ETA/estimated time of arrival, key contact information, potential payor source information, the ability to upload any discharge planning, physician and pharmacy order documentation. According to some embodiments, this can all be done within a matter of approximately ‘6-clicks.’

Once reservation is submitted, the CR technology may also send immediate notification to the CR client facilities admissions department via email, text message, push notifications, notifications that can be received via smartphone devices, or using any other such electronic notification method. This may be in addition to the transmissions via the internal CR software system itself.

The CR solution may enable the automatic transmission of admission confirmation information from the CR client facility to the discharging facility in response to a user input. For example, an employee of the CR client facility may select a button presented by the web-based CR solution that represents that a patient has successfully been admitted, and in response, the system may, for example automatically generate an email or some other form of electronic notification to one or more recipients containing information such as the identification of the patient, the identification of the facility, the date, the time, and other information that may be pertinent to the admission.

The CR solution may also archive all transmissions from the referral sources on behalf of the CR client. The CR solution may also allow the CR client to upload and transmit discharge planning documents to other facilities if and when the patient/resident leaves the CR client facility to go to another type of healthcare provider and/or back to their own residence.

According to some embodiments, it is approximated that the average CR client facility could save upwards of 30 to 50 hours/per week of phone time, by having this CR software solution in place.

In some embodiments, resource management may be implemented with blockchain technology. For example, the process flow of patient transitions between facilities are recorded and identified by using specific blockchain identifiers. The blockchain ledger will be held by nodes (e.g., facilities) and provide for a higher degree of information security than related art systems as it relates to patient health information (PHI).

According to some implementations, there is provided an online platform for connecting a plurality of facilities as it relates to patient transfers from one facility to another. In some cases, a time status of patient discharges and available beds at receiving healthcare facilities will be recorded within the blockchain. As a result, the patient discharge process may be simplified through the online platform.

Furthermore, certain implementations store relevant facility audit information on the blockchain. Each relevant transaction that occurs (e.g., utilizing an audit system) may be analyzed in real-time to immediately identify problem areas in the facility that need to be addressed, whether those problems are procedural in nature or connected to individual equipment, staff, or departments. Moreover, ratings may be recorded on the blockchain and tied to specific transactions. Accordingly, facilities and resources may be relatively and reliably rated for comparison.

Certain embodiments will be described with reference to the figures. One of ordinary skill will recognize that these embodiments are merely examples, and various modifications may be made thereto without departing from the scope of the present disclosure.

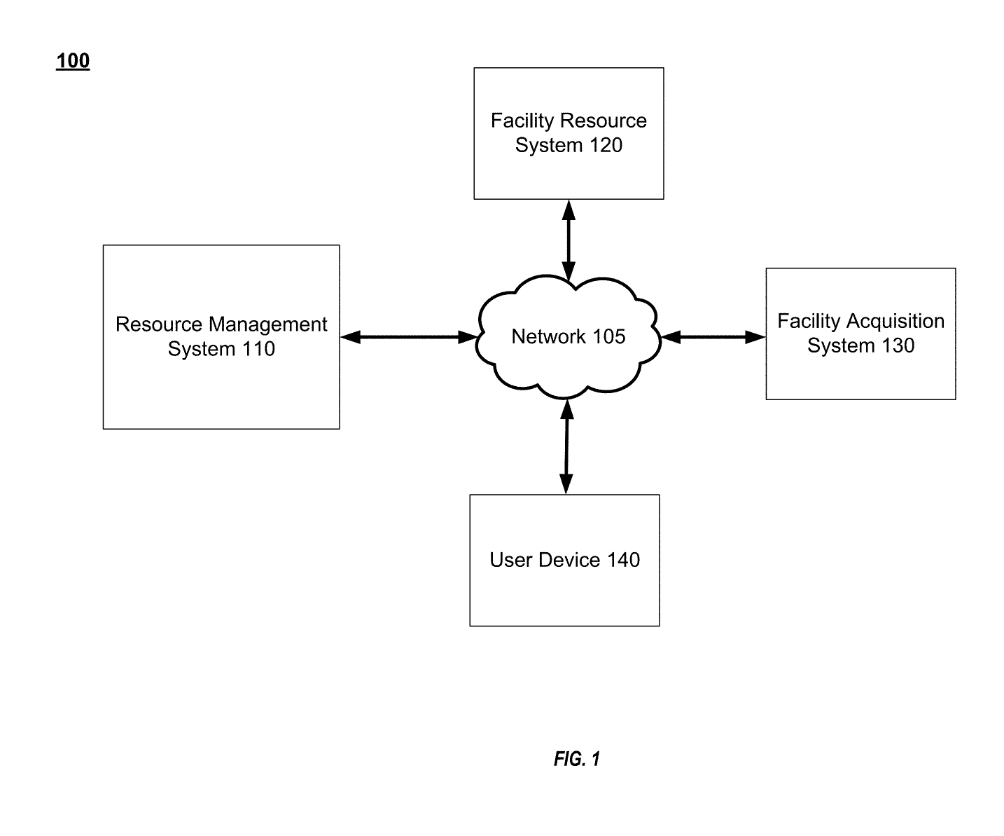

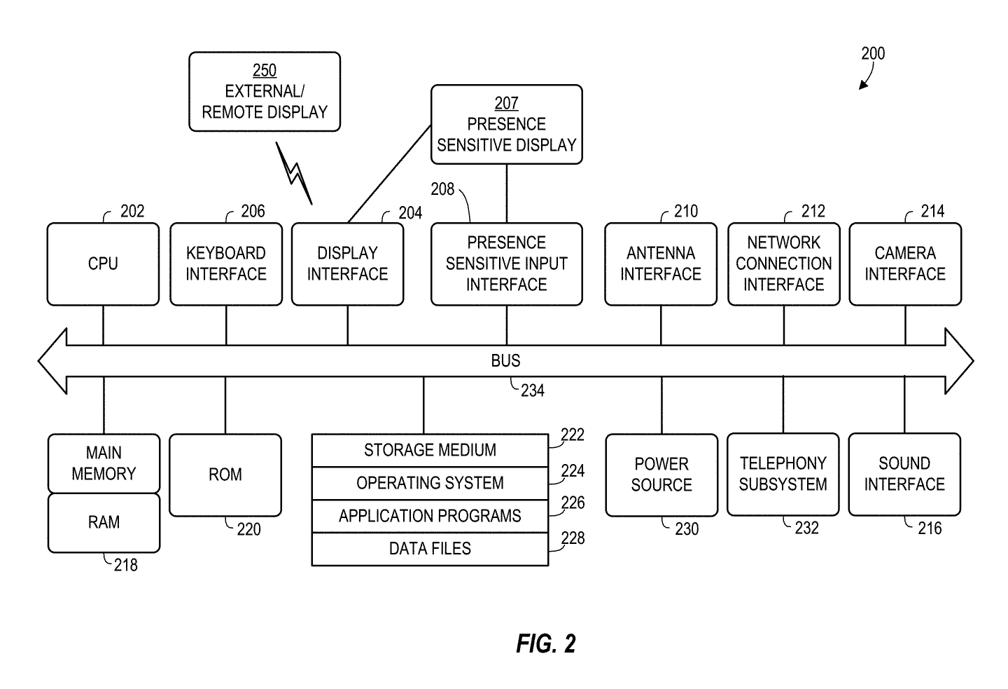

FIG. 1 illustrates an example system environment 100 for implementing certain aspects of the present disclosure. System environment 100 includes a resource management system 110 connected to one or more facility resource systems 120, resource acquisition systems 130, and user devices 140 through network 105. System environment 100 may include a plurality of facility resource systems 120, resource acquisition systems 130, and user devices 140. As will be understood by one of ordinary skill, each of the resource management system 110, facility resource system 120, and resource acquisition system 130 may each include one or more logically or physically distinct systems, and aspects of the disclosure may be implemented in environments with fewer, additional or alternative systems and devices. An example computer architecture that may implement one or more aspects of the system environment 100 is described below with reference to FIG. 2.

Resource management system 110 may provide a platform (e.g., a web-based portal) for acquiring and listing resources from a plurality of facilities (e.g., facility resource systems 120). Facility resource system 120, resource acquisition system 130, and user device 140 may access the web-based portal over network 105. For example, users (e.g., admission coordinators, facility managers, patients) may provide credential information to a web-based portal to have access to the platform. Resource management system 110 may transform data from desperate facilities in order to combine the facility information (e.g., as discussed below in greater detail).

Resource management system 110 receives, from facility resource system 120, facility information of a respective facility. The facility information may include one or more of, for example, resource type (e.g., machines such as IV pumps and breathing assistant machines, staff such as cardiac nurse or physical therapist, services such as wound care and IV antibiotics, beds, and rooms (e.g., private v. shared)), resource availability (e.g., available, unavailable, or retrievable), facility location, facility type (e.g., hospice agency, skilled nursing facility, assisted living, and home health care agency), facility group identifier (e.g., if the facility is part of a facility group), images of the facility and/or services, contact information, accepted insurance (e.g., private insurance, Medicare, and Medicaid), accessibility, rating (e.g., patient rating of facilities and/or resources), and transport services (e.g., to/from hospital).

Resource management system 110 may provide the facility information to the resource acquisition systems 130. Resource management system 110 may organize facility information for a plurality of facilities, for example, based on indications from resource acquisition system 130. For example, resource acquisition system 130 may provide resource requirements (e.g., needed resources for a particular patient). For example, an admission coordinator at a hospital may, through resource acquisition system 130, search for facilities that meet certain criteria (e.g., an assisted living facility within 20 miles of the hospital, having open beds and a cardiac nurse with transportation services). Resource management system 110 may provide resource acquisition system 130 facilities that meet the criteria. In some cases, resource management system 110 may provide the listing to user device 140, which may be a device of a patient. In certain implementations, resource management system 110 may integrate with patient files (e.g., PHI) of resource acquisition system 130 and analyze the same (e.g., using machine learning) to determine relevant facilities for the patient. For example, if a patient has been prescribed occupational therapy, resource management system 110 will only identify facilities that have an occupational therapist on staff In some cases, resource management system 110 may automatically identify potential facilities in response to discharge order being entered into the patient-health information. In certain instances, resource management system 110 may analyze the patient data to anticipate discharge (e.g., by changes to vital signs detected over time in comparison to previously discharged patients). Such enhancements may be provided by, for example, various machine-learning algorithms as would be understood by one of ordinary skill in light of the present disclosure.

Resource acquisition system 130 and/or user device 140 may indicate, to resource management system 110 a selection of one or more facilities for the patient. For example, in response to receiving a list of assisted living facilities that meet the patient or facility requirements, either the patient or an admission coordinator may review the relevant facilities and select a facility. Resource management system 110 may schedule a resource registration at the selected facility (e.g., through facility resource system 120). In some cases, resource management system 110 may integrate with scheduling platforms of the facility resource system 120 and automatically schedule appropriate auxiliary resources (e.g., nurses, machines) in accordance with the patient's needs. In certain instances, resource management system 110 may schedule delivery of needed equipment and/or staff services for a facility group (e.g., if certain facilities share or loan items). In such cases, resource management system may track resource locations and reservations within the facility group and initiate transfer between group facilities. As non-limiting examples, auxiliary resources may include a dialysis technician and a dialysis machine (for a patient needing dialysis), a bi-pap machine (for a patient requiring breathing assistance), a chest draining technician, a cardiac nurse, a peg-tube, wound vacuum and technician, and restraints.

When a reservation is made, resource management system 110 may automatically notify facility resource system 120, resource acquisition system 130, and/or user device 140 (e.g., through email or text messaging) of the schedules resource. In some instances, resource management system 110 may facilitate two-way communication between notify facility resource system 120 and resource acquisition system 130 or user device 140 in response to a reservation. Facilitating two-way communication may include providing, to resource acquisition system 130 or user device 140, links (e.g., in emails or text messages) to facility resource system 120. In some instances, resource management system 110 may initiate a multi-party live chat between facility resource system 120 and resource acquisition system 130 and/or user device 140 when a reservation is selected.

In some instances, resource management system 110 may implement and/or maintain a blockchain of relevant facility and patient data. Certain aspects of the facility and/or patient data may be securely encrypted within the blockchain. For example, individual facilities (e.g., facility resource system 120 and resource acquisition system 130) and/or patients (e.g., user device 140) may have respective public/private key pairs for encryption and data signature. Users (e.g., admission coordinators, facility managers, patients) may be granted access to information on the blockchain by virtue of their login credentials provided through the web-portal. Facility resource system(s) 120 and resource acquisition system(s) 130 may act as nodes on the blockchain. For example, the block chain may securely store and transfer patient information between resource acquisition system 130 and facility resource system 120. In addition, the blockchain may log reservations, reservation details (e.g., length of stay, number of services provided), and/or patient ratings tied to particular reservations. Moreover, by analyzing the reservation data and comparing the same to like-situated facilities, resource management system 110 may provide an objective comparison between facilities. Accordingly, resource management system 110 may provide enhanced and verified resource and/or facility ratings.

As discussed above, facility resource system 120 may provide resource management system 110 facility information. Such resources may be globally applied, and available resources may be determined based on known reservations (e.g., if a facility has 100 beds and 80 are reserved, the facility has 20 beds). In some instances, resource management system 110 may integrate with a resource allocator of facility resource system 120 to dynamically track resource allocation and commitment. In some instances, when a reservation is made, resource management system 110 may interact with the resource allocator of facility resource system 120 to automatically modify available resources (e.g., by indicating an additional bed is reserved) and/or schedule staffing or other required resources. A user of facility resource system 120 may list and/or update facility resources through the web-portal.

Network 105 may be of any suitable type, including individual connections via the internet such as cellular or Wi-Fi networks. In some embodiments, network 105 may connect terminals using direct connections such as radio-frequency identification (RFID), near-field communication (NFC), Bluetooth™, low-energy Bluetooth™ (BLE), Wi-Fi™, ZigBee™, ambient backscatter communications (ABC) protocols, USB, or LAN. Because the information transmitted may be personal or confidential, security concerns may dictate one or more of these types of connections be encrypted or otherwise secured. In some embodiments, however, the information being transmitted may be less personal, and therefore the network connections may be selected for convenience over security.

FIG. 2 is a block diagram of an illustrative computing device architecture 200, according to an example implementation. The computing device architecture 200 may be used to implement the CR solution or one or more components of the CR solution according to some example embodiments. For example, computer device architecture 200 may be used to implement one or more of resource management system 110, facility resource system 120, resource acquisition system 130, and/or user device 140. It will be understood that the computing device architecture 200 is provided for example purposes only and does not limit the scope of the various implementations of the present disclosure. In some embodiments, the CR solution or one or more components of the CR solution may have fewer, alternative, or additional components as that illustrated in FIG. 2.

The computing device architecture 200 of FIG. 2 includes a central processing unit (CPU) 202, where computer instructions are processed, a display interface 204 that acts as a communication interface and provides functions for rendering video, graphics, images, and texts on the display. In certain example implementations of the disclosed technology, the display interface 204 may be directly connected to a local display, such as a touch-screen display associated with a mobile computing device. In another example implementation, the display interface 204 may be configured for providing data, images, and other information for an external/remote display 250 that is not necessarily physically connected to the mobile computing device. For example, a desktop monitor may be used for mirroring graphics and other information that is presented on a mobile computing device. In certain example implementations, the display interface 204 may wirelessly communicate, for example, via a Wi-Fi channel or other available network connection interface 212 to the external/remote display 250.

In an example implementation, the network connection interface 212 may be configured as a communication interface and may provide functions for digital virtual assistant using voice, rendering video, graphics, images, text, other information, or any combination thereof on the display. In one example, a communication interface may include a microphone, camera, serial port, a parallel port, a general-purpose input and output (GPIO) port, a game port, a universal serial bus (USB), a micro-USB port, a high definition multimedia (HDMI) port, a video port, an audio port, a Bluetooth port, a near-field communication (NFC) port, another like communication interface, or any combination thereof. In one example, the display interface 204 may be operatively coupled to a local display, such as a touch-screen display associated with a mobile device or voice enabled device. In another example, the display interface 204 may be configured to provide video, graphics, images, text, other information, or any combination thereof for an external/remote display 250 that is not necessarily connected to the mobile computing device. In one example, a desktop monitor may be used for mirroring or extending graphical information that may be presented on a mobile device. In another example, the display interface 204 may wirelessly communicate, for example, via the network connection interface 212 such as a Wi-Fi transceiver to the external/remote display 250.

The computing device architecture 200 may include a keyboard interface 206 that provides a communication interface to a keyboard. In one example implementation, the computing device architecture 200 may include a presence sensitive input interface 208 for connecting to a presence sensitive display 207. According to certain example implementations of the disclosed technology, the presence sensitive input interface 208 may provide a communication interface to various devices such as a pointing device, a touch screen, a depth camera, microphone, etc. which may or may not be associated with a display.

The computing device architecture 200 may be configured to use an input device via one or more of input/output interfaces (for example, the keyboard interface 206, the display interface 204, the presence sensitive input interface 208, network connection interface 212, camera interface 214, sound interface 216, etc.) to allow a user to capture information into the computing device architecture 200. The input device may include a mouse, a trackball, a directional pad, a track pad, a touch-verified track pad, a presence-sensitive track pad, a presence-sensitive display, a scroll wheel, a digital camera, a digital video camera, a web camera, a microphone, a sensor, a smartcard, and the like. Additionally, the input device may be integrated with the computing device architecture 200 or may be a separate device. For example, the input device may be an accelerometer, a magnetometer, a digital camera, a microphone, and an optical sensor.

Example implementations of the computing device architecture 200 may include an antenna interface 210 that provides a communication interface to an antenna; a network connection interface 212 that provides a communication interface to a network. As mentioned above, the display interface 204 may be in communication with the network connection interface 212, for example, to provide information for display on a remote display that is not directly connected or attached to the system. In certain implementations, camera interface 214 acts as a communication interface and provides functions for capturing digital images from a camera. In certain implementations, a sound interface 216 is provided as a communication interface for converting sound into electrical signals using a microphone and for converting electrical signals into sound using a speaker. According to example implementations, a random-access memory (RAM) 218 is provided, where computer instructions and data may be stored in a volatile memory device for processing by the CPU 202.

According to an example implementation, the computing device architecture 200 includes a read-only memory (ROM) 220 where invariant low-level system code or data for basic system functions such as basic input and output (I/O), startup, or reception of keystrokes from a keyboard are stored in a non-volatile memory device. According to an example implementation, the computing device architecture 200 includes a storage medium 222 or other suitable type of memory (e.g. such as RAM, ROM, programmable read-only memory (PROM), erasable programmable read-only memory (EPROM), electrically erasable programmable read-only memory (EEPROM), magnetic disks, optical disks, floppy disks, hard disks, removable cartridges, flash drives), where the files include an operating system 224, application programs 226 (including, for example, a web browser application, a widget or gadget engine, and or other applications, as necessary) and data files 228 are stored. According to an example implementation, the computing device architecture 200 includes a power source 230 that provides an appropriate alternating current (AC) or direct current (DC) to power components.

According to an example implementation, the computing device architecture 200 includes a telephony subsystem 232 that allows the computing device to transmit and receive sound over a telephone network. The constituent devices and the CPU 202 communicate with each other over a bus 234.

According to an example implementation, the CPU 202 has appropriate structure to be a computer processor. In one arrangement, the CPU 202 may include more than one processing unit. The RAM 218 interfaces with the computer BUS 234 to provide quick RAM storage to the CPU 202 during the execution of software programs such as the operating system application programs, and device drivers. More specifically, the CPU 202 loads computer-executable process steps from the storage medium 222 or other media into a field of the RAM 218 to execute software programs. Data may be stored in the RAM 218, where the data may be accessed by the computer CPU 202 during execution.

The storage medium 222 itself may include a number of physical drive units, such as a redundant array of independent disks (RAID), a floppy disk drive, a flash memory, a USB flash drive, an external hard disk drive, thumb drive, pen drive, key drive, a High-Density Digital Versatile Disc (HD-DVD) optical disc drive, an internal hard disk drive, a Blu-Ray optical disc drive, or a Holographic Digital Data Storage (HDDS) optical disc drive, an external mini-dual in-line memory module (DIMM) synchronous dynamic random access memory (SDRAM), or an external micro-DIMM SDRAM. Such computer readable storage media allow a computing device to access computer-executable process steps, application programs and the like, stored on removable and non-removable memory media, to off-load data from the device or to upload data onto the device. A computer program product, such as one utilizing a communication system may be tangibly embodied in storage medium 222, which may include a machine-readable storage medium.

According to one example implementation, the term computing device, as used herein, may be a CPU, or conceptualized as a CPU (for example, the CPU 202 of FIG. 2). In this example implementation, the computing device (CPU) may be coupled, connected, and/or in communication with one or more peripheral devices, such as display. In another example implementation, the term computing device, as used herein, may refer to a mobile computing device such as a smart phone, tablet computer, or smart watch. In this example implementation, the computing device may output content to its local display and/or speaker(s). In another example implementation, the computing device may output content to an external display device (e.g., over Wi-Fi) such as a TV or an external computing system.

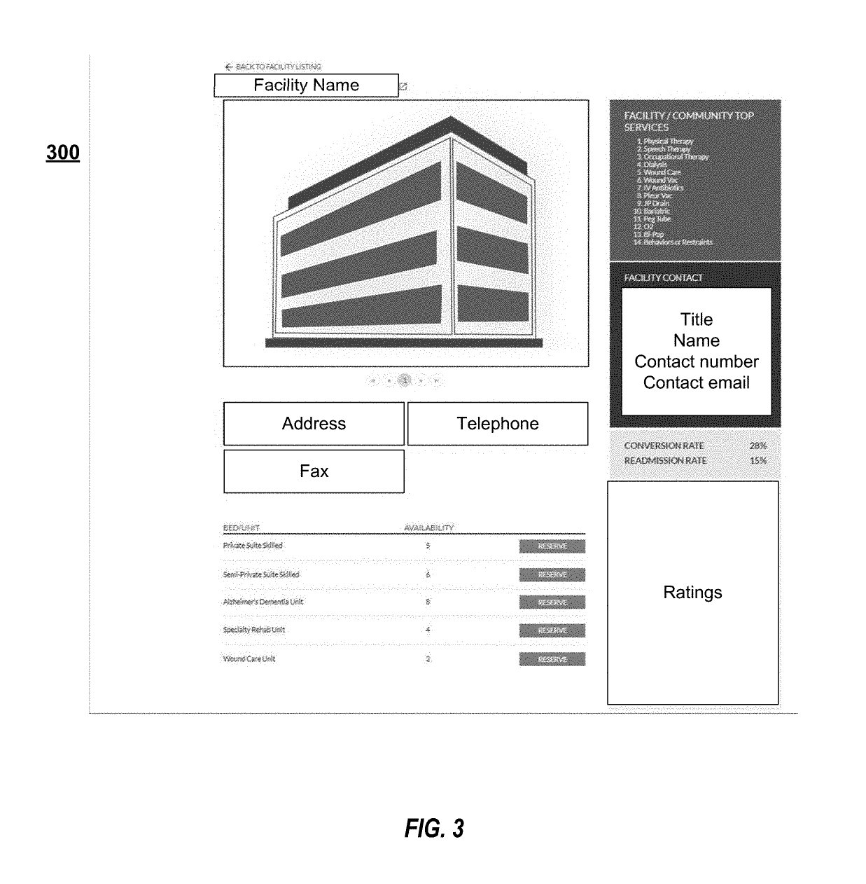

FIG. 3 illustrates a facility resource page 300 according to an example embodiment. The facility resource page 300 includes a facility name, facility image(s) facility contact information, facility services, facility rankings, facility metrics, and facility resources. The facility resource page 300 may be provided to, for example, resource acquisition system 130 and/or user device 140 through a web-based solution hosted by resource management system 110. For example, after a facility is selected from among a plurality of potential facilities (i.e., potential transfer facilities for the patient), resource management system 110 may provide facility resource page 300 to resource acquisition system 130 and/or user device 140. Resource acquisition system 130 and/or user device 140 may indicate a selection of a resource (i.e., a user selection), and resource management system 110 may automatically schedule the resource for the patient. One of ordinary skill will understand that a facility resource page 300 may have fewer, additional, or alternative fields while remaining within the scope of the present disclosure.

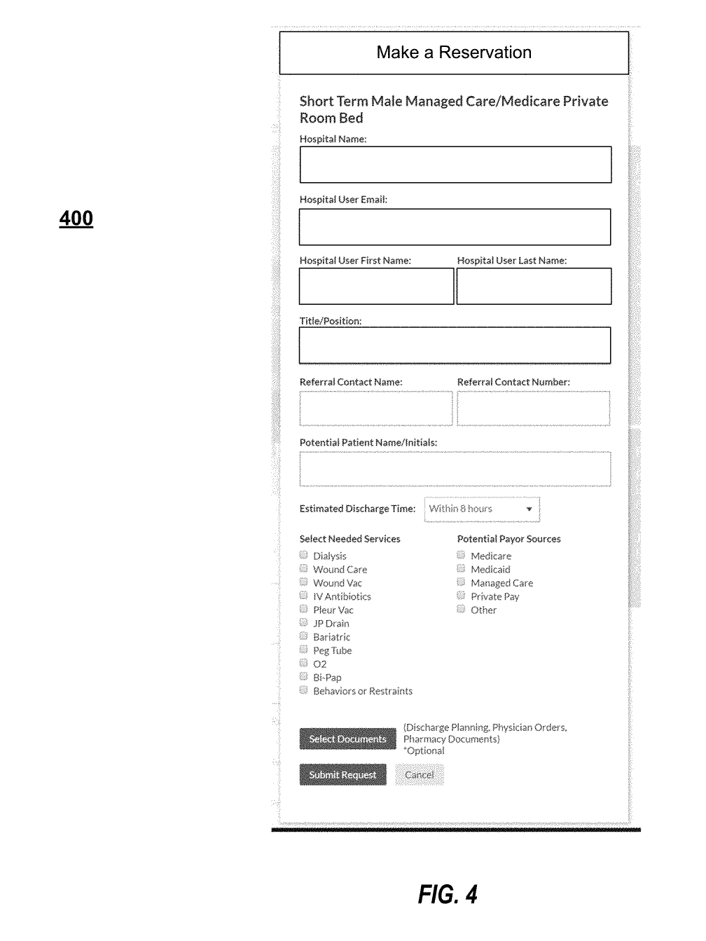

FIG. 4 illustrates a reservation interface 400 according to an example embodiment. The reservation interface includes text fields for a hospital name, hospital contact information (e.g., email, first and last name, title), referral names and contacts, and patient identifier (e.g., name or initials), a dropdown menu of estimated discharge times (e.g., 8 hours, 2 days, etc.), selectable listings of needed services (e.g., auxiliary resources for the patient) and payment sources (e.g., Medicare, Medicaid, private insurance, self-pay, or other), and an upload field for uploading relevant patient documents (e.g., discharge planning, physician orders, prescriptions). A user of resource acquisition system 130 (e.g., an admission coordinator) may enter the patient information into reservation interface 400 and submit a request for the resource. Resource management system 110 may then identify one or more facilities corresponding to facility resource system 120 capable of providing the identified resource and needed services for the patient at the identified discharge time. One of ordinary skill will understand that a reservation interface 400 may have fewer, additional, or alternative fields while remaining within the scope of the present disclosure.

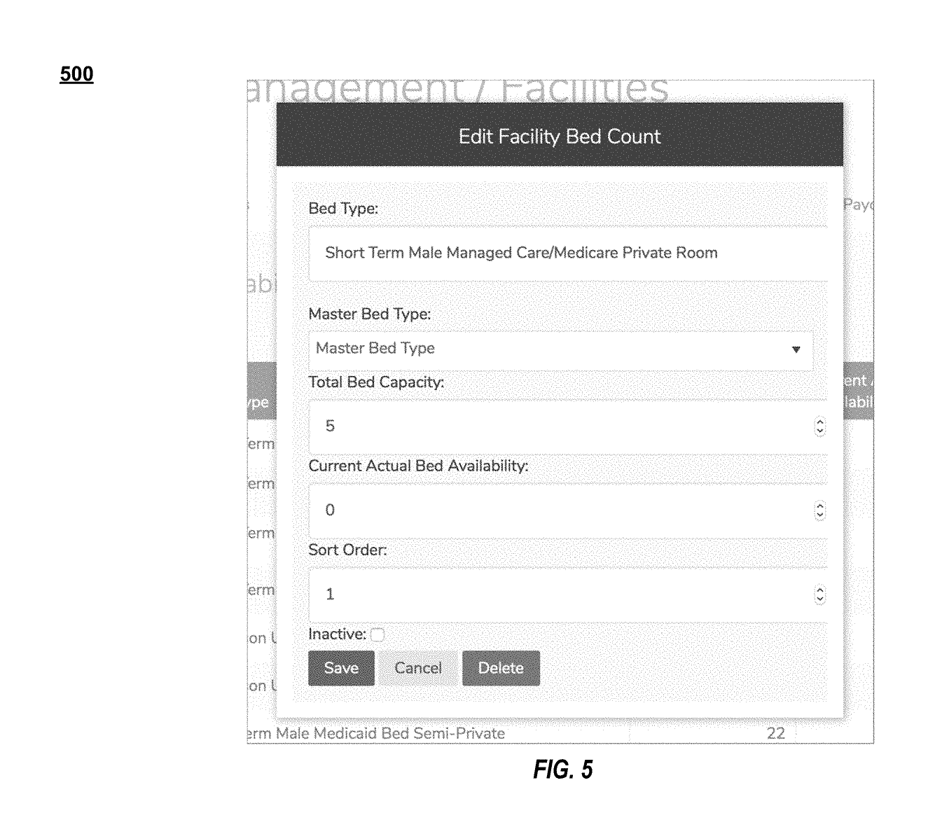

FIG. 5 illustrates a resource management interface 500 according to an example embodiment. Resource management interface 500 may be provided to facility resource system 120 to modify resources available for reservation. Resource management interface 500 includes general resource type (e.g., bed type), a resource type (e.g., master bed type), resource capacity (e.g., a number of the resource at the facility), current availability (e.g., a number of the resources currently available for reservation), and a sort order (e.g., a ranking of the resource from among a plurality of resources provided by the facility). One of ordinary skill will understand that a resource management interface 500 may have fewer, additional, or alternative fields while remaining within the scope of the present disclosure.

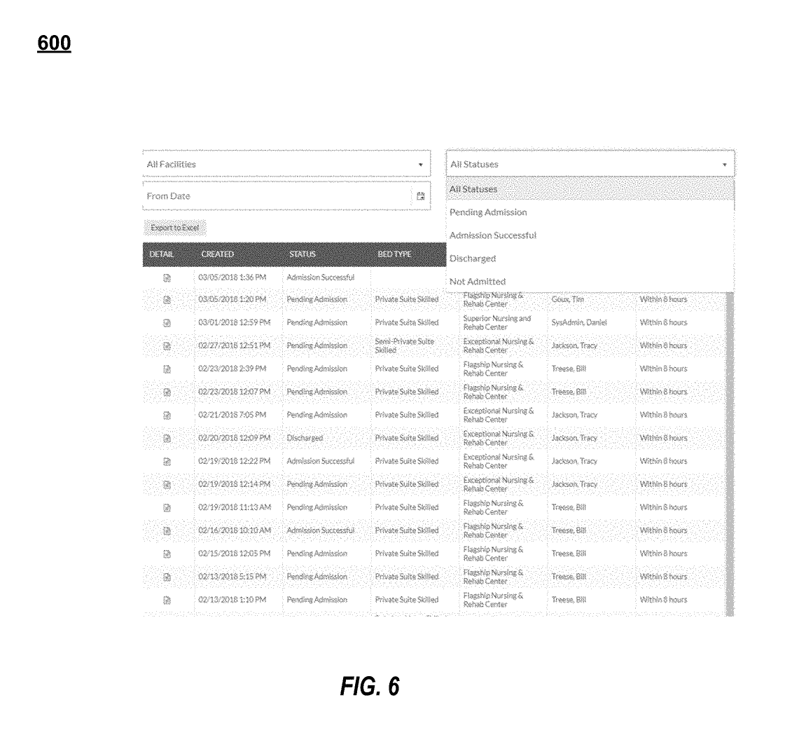

FIG. 6 illustrates a reservation tracker 600 according to an example embodiment. Reservation tracker 600 may be resource acquisition system 130 to review current and past reservation requests. Reservation tracker 600 may include various search and/or limiting fields (e.g., facility identifiers, dates, reservation status) as well as listing of specific resource reservations. The resource reservations may be provided in a list identified date created, status, bed type, facility name, patient, and discharge time. One of ordinary skill will understand that a reservation tracker 600 may have fewer, additional, or alternative fields while remaining within the scope of the present disclosure.

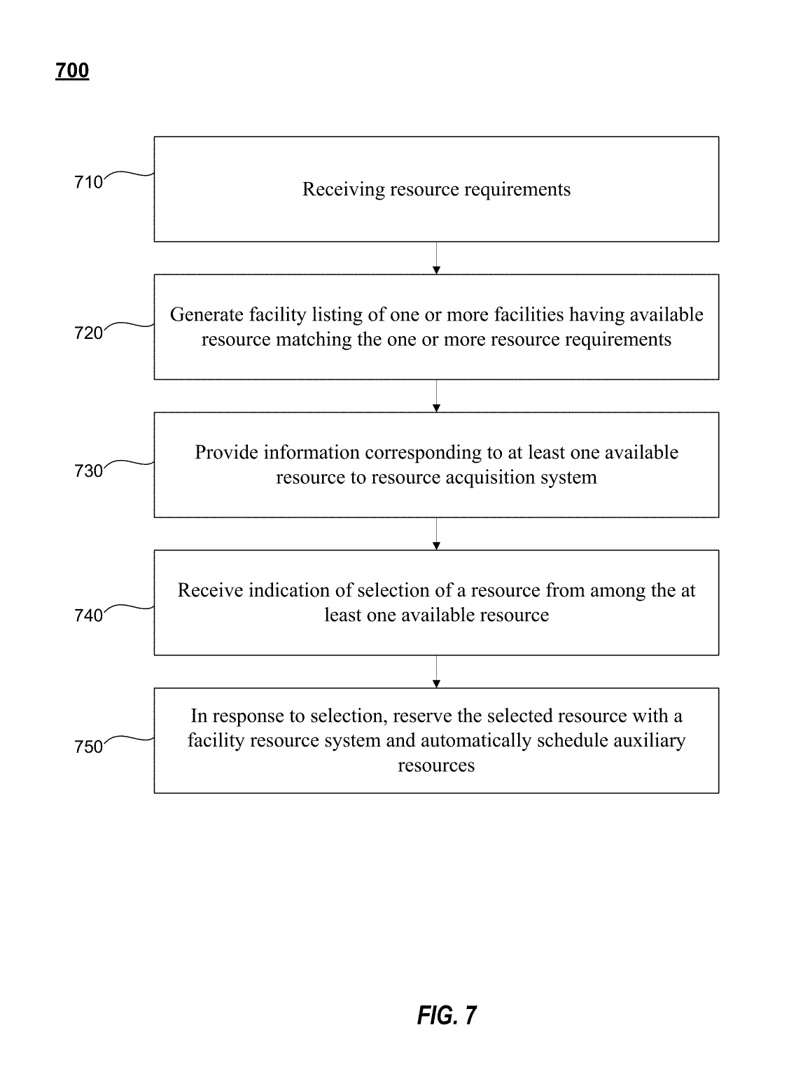

FIG. 7 is a flowchart of an example method 700 according to an example embodiment. Method 700 may be performed, at least in part, by resource management system 110. Referring to FIG. 7, resource management system 110 receives 710 one or more resource requirements. The more resource requirements may include at least one from among a resource type, facility location, facility type, rating, and transport services. In some cases, the one or more resource requirements may include a service needed by a patient (e.g., dialysis, wound care, wound vac, IV antibiotics, Pleur vac, JP drain, bariatric pressure monitoring, peg tube, oxygen, bi-pap machine, and/or restraints).

Resource management system 110 generates 720 a facility listing identifying one or more facilities with available resource matching the resource requirements. For example, if the patient requires dialysis, the identified facilities would have to have a dialysis machine and/or be able to obtain a dialysis machine. Resource management system 110 outputs 730 the facility listing (e.g., to resource acquisition system 130 and/or user device 140). For example, the facility listing may be provided via a web-portal, over email, or through text messaging. Resource management system 110 receives 740 a selection of a resource/facility from among the listing. The selection may be received in a similar manner in which the facility listing was provided.

Resource management system 110 reserves 750 the selected resource and automatically schedules 760 auxiliary resource(s). The auxiliary resource may include at least one of a specialized device (dialysis machine) and a specialized staff (e.g., nurse, therapist or technician). Automatically scheduling 760 one or more auxiliary resources may include identifying one or more specialized staff necessary for the resource, accessing an employee scheduling platform of the selected facility (e.g., facility resource system 120), and scheduling the identified specialized staff to coincide with the resource reservation.

In some instances, resource management system 110 may modify the selected facility's resource records in response to the selection. For example, resource management system may access facility resource system 120 to change an availability indicator of the scheduled resource and auxiliary resources at the facility resource system 120 to unavailable.

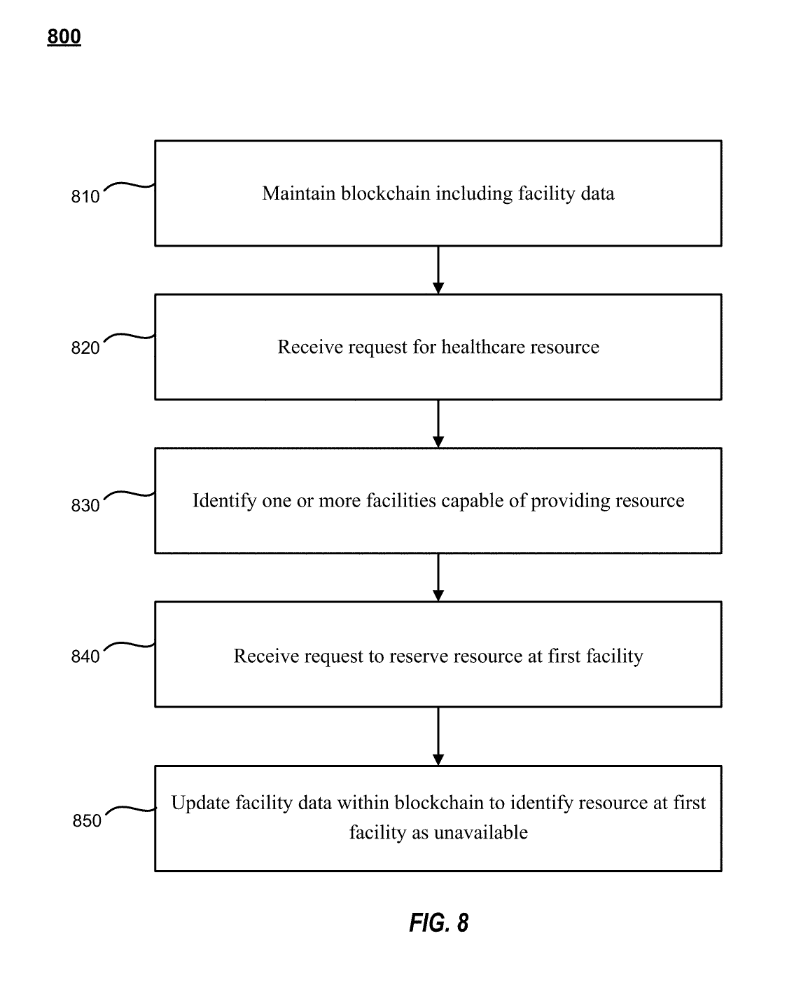

FIG. 8 is a flowchart of an example method 800 according to an example embodiment. Method 800 may be performed, at least in part, by resource management system 110. Referring to FIG. 8, resource management system 110 maintains 810 a blockchain that stores facility data for a plurality of facilities. The facility data may be provided by facility resource system(s) 120 and/or derived from external systems.

Resource management system 110 receives 820 a request for a healthcare resource. Resource management system 110 identifies 830 one or more facilities capable of providing the requested healthcare resource. The facilities may be identified 820 based on the facility data. In some cases, resource management system 110 may receive 820 the request in the form of a discharge order for a patient (e.g., from resource acquisition system 130), and automatically identify 830 the one or more facilities based on the discharge order. Resource management system 110 may identify 830 facilities based on a plurality of criteria (e.g., required patient services). In some cases, the criteria may be identified by analyzing the discharge order (e.g., through machine learning or natural language processing). The criteria may include criteria one or more of a geographic region, an accepted insurance, a needed service, and needed dates. In some instances, resource management system 110 may have access to patient health information stored by resource acquisition system 130, and may monitor the patient information to detect a discharge order for the patient.

Resource management system 110 receives 840 a request to reserve the healthcare resource at a first facility among the identified facilities. Resource management system 110 receives 850 then schedules the resource reservation by, for example, updating the facility data within the blockchain to identify the healthcare resource at the first facility as unavailable. The reservation information may include a plurality of identifiers indicative of the patient, the first facility, and the reserved healthcare resource. In some cases, the blockchain stores reservation information, and resource management system 110 may update the reservation information within blockchain to indicate that the patient has reserved the healthcare resource at the first facility.

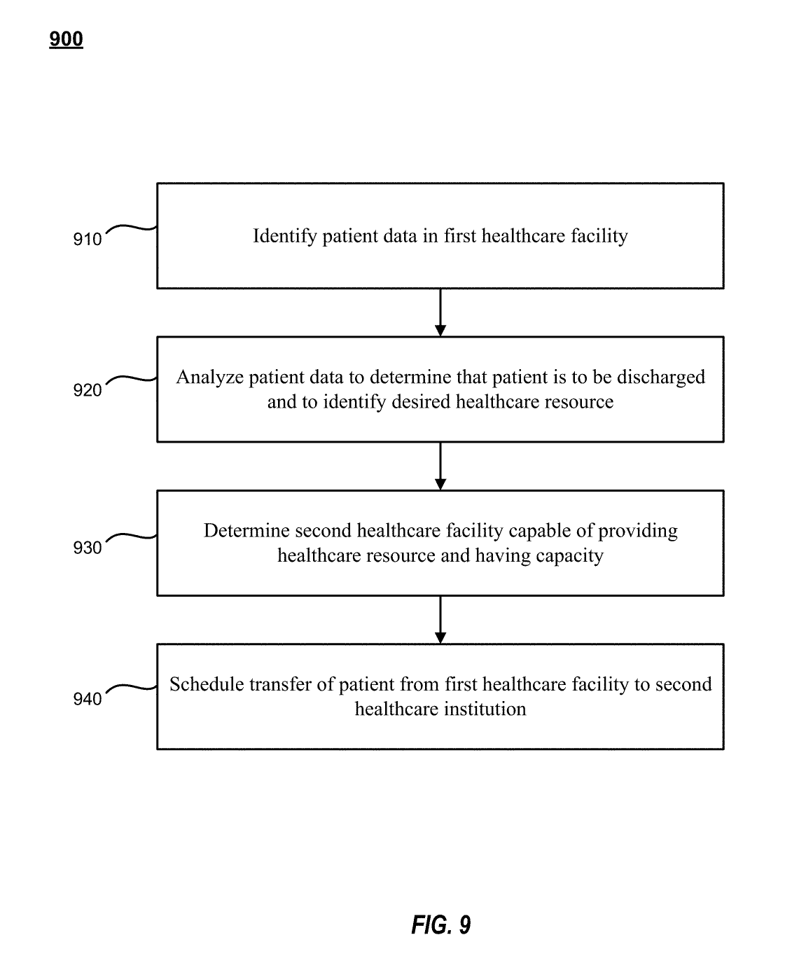

FIG. 9 is a flowchart of an example method 900 according to an example embodiment. Method 900 may be performed, at least in part, by resource management system 110. Referring to FIG. 9, resource management system 110 identifies 910 patient data in first healthcare facility (e.g., resource acquisition system 130). The patient data may correspond to an admitted patient at the first healthcare facility.

Resource management system 110 analyzes 920 the patient data to determine that the patient is to be discharged from the first healthcare facility and to identify a healthcare resource for the patient post-discharge. In some cases, resource management system 110 identifies a discharge order for the patient from within the patient data, and analyzes 920 the discharge order to determine the requisite healthcare resource (e.g., using machine learning and/or natural language processing). In some implementations, resource management system 110 may analyze 920 the patient data to determine a patient recovery trajectory and determine expected required healthcare resources prior to receiving a discharge order (e.g., based on previous needed resources for similarly situated patients). Resource management system 110 determines 930 a second healthcare facility capable of providing the identified healthcare resource and having capacity for the patient.

Resource management system 110 schedules 940 patient transfer patient from the first healthcare facility the second healthcare facility. Scheduling 940 the patient transfer may include reserving the identified healthcare resource at the second healthcare facility (e.g., facility resource system 120) for the patient. In some cases, scheduling 940 the transfer of the patient may further include accessing a staff scheduling platform hosted by the second healthcare facility, and scheduling one or more staff members at the second healthcare facility to during the transfer. In some instances, scheduling 940 the patient transfer includes a staff scheduling platform hosted by the second healthcare facility, and assigning one or more healthcare machines required healthcare resource to the patient during the transfer.

In example implementations of the disclosed technology, a computing device may include any number of hardware and/or software applications that are executed to facilitate any of the operations. In example implementations, one or more I/O interfaces may facilitate communication between the computing device and one or more input/output devices. For example, a universal serial bus port, a serial port, a disk drive, a CD-ROM drive, and/or one or more user interface devices, such as a display, keyboard, keypad, mouse, control panel, touch screen display, microphone, etc., may facilitate user interaction with the computing device. The one or more I/O interfaces may be used to receive or collect data and/or user instructions from a wide variety of input devices. Received data may be processed by one or more computer processors as desired in various implementations of the disclosed technology and/or stored in one or more memory devices.

One or more network interfaces may facilitate connection of the computing device inputs and outputs to one or more suitable networks and/or connections; for example, the connections that facilitate communication with any number of sensors associated with the system. The one or more network interfaces may further facilitate connection to one or more suitable networks; for example, a local area network, a wide area network, the Internet, a cellular network, a radio frequency network, a Bluetooth enabled network, a Wi-Fi enabled network, a satellite-based network any wired network, any wireless network, etc., for communication with external devices and/or systems.

In the preceding description and accompanying slides, numerous specific details are set forth. It is to be understood, however, that implementations of the disclosed technology may be practiced without these specific details. In other instances, well-known methods, structures and techniques have not been shown in detail in order not to obscure an understanding of this description. References to “one implementation,” “an implementation,” “example implementation,” “various implementations,” etc., indicate that the implementation(s) of the disclosed technology so described may include a particular feature, structure, or characteristic, but not every implementation necessarily includes the particular feature, structure, or characteristic. Further, repeated use of the phrase “in one implementation” does not necessarily refer to the same implementation, although it may.

Throughout the specification and the claims, the following terms take at least the meanings explicitly associated herein, unless the context clearly dictates otherwise. The term “connected” means that one function, feature, structure, or characteristic is directly joined to or in communication with another function, feature, structure, or characteristic. The term “coupled” means that one function, feature, structure, or characteristic is directly or indirectly joined to or in communication with another function, feature, structure, or characteristic. The term “or” is intended to mean an inclusive “or.” Further, the terms “a,” “an,” and “the” are intended to mean one or more unless specified otherwise or clear from the context to be directed to a singular form.

As used herein, unless otherwise specified the use of the ordinal adjectives “first,” “second,” “third,” etc., to describe a common object, merely indicate that different instances of like objects are being referred to, and are not intended to imply that the objects so described must be in a given sequence, either temporally, spatially, in ranking, or in any other manner.

Certain implementations of the disclosed technology have been described above or shown in the accompanying figures with reference to block and flow diagrams of systems and methods and/or computer program products according to example implementations of the disclosed technology. It will be understood that one or more blocks of the block diagrams and flow diagrams, and combinations of blocks in the block diagrams and flow diagrams, respectively, can be implemented by computer-executable program instructions. Likewise, some blocks of the block diagrams and flow diagrams may not necessarily need to be performed in the order presented, or may not necessarily need to be performed at all, according to some implementations of the disclosed technology.

These computer-executable program instructions may be loaded onto a general-purpose computer, a special-purpose computer, a processor, or other programmable data processing apparatus to produce a particular machine, such that the instructions that execute on the computer, processor, or other programmable data processing apparatus create means for implementing one or more functions specified in the flow diagram block or blocks. These computer program instructions may also be stored in a computer-readable memory that can direct a computer or other programmable data processing apparatus to function in a particular manner, such that the instructions stored in the computer-readable memory produce an article of manufacture including instruction means that implement one or more functions specified in the sequence diagram block or blocks.

Implementations of the disclosed technology may provide for a computer program product, includes a computer-usable medium having a computer-readable program code or program instructions embodied therein, said computer-readable program code adapted to be executed to implement one or more functions specified in the sequence diagram block or blocks. The computer program instructions may also be loaded onto a computer or other programmable data processing apparatus to cause a series of operational elements or steps to be performed on the computer or other programmable apparatus to produce a computer-implemented process such that the instructions that execute on the computer or other programmable apparatus provide elements or steps for implementing the functions specified in the flow diagram block or blocks.

Accordingly, blocks of the block diagrams and flow diagrams support combinations of means for performing the specified functions, combinations of elements or steps for performing the specified functions and program instruction means for performing the specified functions. It will also be understood that each block of the block diagrams and flow diagrams, and combinations of blocks in the block diagrams and flow diagrams, can be implemented by special-purpose, hardware-based computer systems that perform the specified functions, elements or steps, or combinations of special-purpose hardware and computer instructions.

While certain implementations of the disclosed technology have been described in connection with what is presently considered to be the most practical and various implementations, it is to be understood that the disclosed technology is not to be limited to the disclosed implementations, but on the contrary, is intended to cover various modifications and equivalent arrangements included within the scope of the appended claims. Although specific terms are employed herein, they are used in a generic and descriptive sense only and not for purposes of limitation.

This written description uses examples to disclose certain implementations of the disclosed technology, including the best mode, and also to enable any person of ordinary skill to practice certain implementations of the disclosed technology, including making and using any devices or systems and performing any incorporated methods. The patentable scope of certain implementations of the disclosed technology is defined in the claims, and may include other examples that occur to those of ordinary skill. Such other examples are intended to be within the scope of the claims if they have structural elements that do not differ from the literal language of the claims, or if they include equivalent structural elements with insubstantial differences from the literal language of the claims.

What is claimed is:

1. A system comprising:
at least one processor; and
at least one memory having stored thereon computer program code that, when executed by the at least one processor, controls the at least one processor to:
provide, to a resource acquisition system, information corresponding to at least one available resource;
receive an indication of a selection of a resource from among the at least one available resource; and
in response to receiving the selection, reserve the selected resource with a facility resource system and automatically schedule one or more auxiliary resources for the selected resource.

2. The system of claim 1, wherein the computer program code, when executed by the at least one processor, further controls the at least one processor to:
receive, from the resource acquisition system, one or more resource requirements; and
generate a facility listing of one or more facilities having an available resource matching the one or more resource requirements.

3. The system of claim 2, wherein the one or more resource requirements comprises at least one from among a resource type, facility location, facility type, rating, and transport services.

4. The system of claim 2, wherein
the one or more resource requirements comprises a needed service, and
the auxiliary resource comprises at least one of a specialized device and a specialized staff.

5. The system of claim 4, wherein the needed service comprises dialysis, and the auxiliary resource comprises a dialysis technician and a dialysis machine.

6. The system of claim 1, wherein
the computer program code, when executed by the at least one processor, further controls the at least one processor to receive, from the resource acquisition system, a required service; and
automatically scheduling one or more auxiliary resources comprises:
identifying one or more specialized staff necessary for the required service;
accessing an employee scheduling platform of the facility resource system; and
scheduling the identified specialized staff to coincide with the selected resource reservation.

7. The system of claim 1, wherein the computer program code, when executed by the at least one processor, further controls the at least one processor to:
provide the information corresponding to the at least one available resource to a user device associated with a user requiring the resource; and
receive the indication of the selection of the resource from the user device.

8. The system of claim 1, wherein the computer program code, when executed by the at least one processor, further controls the at least one processor to modify, within the facility resource system, an availability indicator of the scheduled resource and auxiliary resources at the facility resource system.

9. A system comprising:
at least one processor; and
at least one memory having stored thereon computer program code that, when executed by the at least one processor, controls the at least one processor to:
maintain a blockchain including facility data of a plurality of facilities;
receive, from a resource acquisition system, a request for a healthcare resource;
access the facility data to identify one or more facilities from among the plurality of facilities capable of providing the requested healthcare resource;
receive an indication of a request to reserve the healthcare resource at a first facility among the one or more facilities; and
update the facility data within the blockchain to identify the healthcare resource at the first facility as unavailable.

10. The system of claim 9, wherein
the blockchain further stores reservation information,
the request to reserve the healthcare resource comprises an indication of a patient, and
the computer program code, when executed by the at least one processor, further controls the at least one processor to update the reservation information within blockchain to indicate that the patient has reserved the healthcare resource at the first facility.

11. The system of claim 10, wherein the reservation information comprises a plurality of identifiers indicative of the patient, the first facility, and the reserved healthcare resource.

12. The system of claim 9, wherein
the blockchain stores patient health information of a patient a healthcare facility associated with the resource acquisition system, and
the computer program code, when executed by the at least one processor, further controls the at least one processor to:
receive, from the healthcare facility, a discharge order for the patient; and
automatically identify the one or more facilities from among the plurality of facilities capable of providing the requested healthcare resource based on the discharge order.

13. The system of claim 12, wherein the computer program code, when executed by the at least one processor, further controls the at least one processor to update the blockchain to automatically identify the one or more facilities based on the discharge order by identifying, within the discharge order, a plurality of criteria.

14. The system of claim 13, wherein the plurality of criteria comprises at least one from among a geographic region, an accepted insurance, a needed service, and needed dates.

15. A system comprising:
at least one processor; and
at least one memory having stored thereon computer program code that, when executed by the at least one processor, controls the at least one processor to:
identify, at a first healthcare facility, patient data corresponding to a first admitted patient;
analyze the patient data to determine that the patient is to be discharged from the first healthcare facility and to identify a healthcare resource for the patient post-discharge;
determine a second healthcare facility capable of providing the identified healthcare resource and having capacity for the patient; and
schedule transfer of the patient from the first healthcare facility the second healthcare facility.

16. The system of claim 15, wherein the computer program code, when executed by the at least one processor, further controls the at least one processor to identify a discharge order for the patient from within the patient data, and process the discharge order to determine the healthcare resource.

17. The system of claim 15, wherein the computer program code, when executed by the at least one processor, further controls the at least one processor to process the patient data to determine a patient recovery trajectory and determine the healthcare resource prior to receiving a discharge order.

18. The system of claim 15, wherein scheduling the transfer of the patient comprises reserving the identified healthcare resource at the second healthcare facility for the patient.

19. The system of claim 15, wherein scheduling the transfer of the patient comprises accessing a staff scheduling platform hosted by the second healthcare facility, and scheduling one or more staff members at the second healthcare facility to during the transfer.

20. The system of claim 15, wherein scheduling the transfer of the patient comprises accessing a staff scheduling platform hosted by the second healthcare facility, and assigning one or more healthcare machines required healthcare resource to the patient during the transfer.
